(19) United States
(12) Patent Application Publication
(10) Pub. No.: US 2002/0193879 A1
(43) Pub. Date: Dec. 19, 2002

(54) Medical devices having antimicrobial properties

(76) Inventors: Edmund V. Seder, Santa Barbara, CA (US); Jesse N. Nelson, Goleta, CA (US)

Correspondence Address: Marvin E. Jacobs, KOPPEL & JACOBS, 2151 Alessandro Drive, Suite 215, Ventura, CA, 93001, US

(21) Appl. No.: 09/833,961

(22) Filed: Apr. 11, 2001

(51) Int. Cl. A61F 2/20

(52) U.S. Cl. 623/9; 623/23.71

(57) ABSTRACT

Microbial growth on a first surface of a medical device in indirect contact with tissue such as a prosthesis and particularly a valve, cartridge or ring in a voice prosthesis, is inhibited by providing antimicrobial activity at a level sufficient to retard growth of a microbial film by dispersing an antimicrobial agent such as triclosan or butyl paraben dispersed in a medical grade silicone elastomer. The valve, ring or cartridge is in contact with body fluids containing microorganisms and nutrients therefor. The antimicrobial surface can interfere with or inhibit the growth of a biofilm, bacterial layer or a yeast layer. The body of the prosthesis may also contain an antimicrobial surface as long as it is non-toxic to the tissue it contacts.

### TECHNICAL FIELD

The present invention relates to microbial-resistant medical devices and, more particularly, this invention relates to a voice prosthesis having a valve which retards growth of microbial organisms.

### BACKGROUND OF THE INVENTION

Medical devices, particularly synthetic resin prosthetic devices which are used in environments where micro-organisms such as fungi or yeast and/or bacteria are actively growing, can become covered with a biofilm colonized layer to the point where the function of the device is impaired. After growth of the biofilm microbial layer, filaments can grow and descend into the body or wall of the polymeric device and detrimentally affect its physical properties until the device no longer functions. The fouled device must be cleaned or discarded.

Whenever a prosthesis is in contact with moisture in a warm, dark environment, the surfaces are subject to microbial growth, usually containing a predominant amount of Candida usually mixed with bacteria. The microbial growth can interfere with the functioning of the prosthesis, requiring removal of the prosthesis for disposal or cleaning. The microbial growth is a persistent problem in the management and care of patients who have had their larynx removed and utilize a voice prosthesis since the prosthesis is exposed to a non-sterile, humid, warm, nutrient rich environment.

There are several options for restoring speech to patients who have had their larynx removed. One procedure is to surgically create a puncture or fistula between the trachea and the esophagus. A tracheoesophageal voice prosthesis containing a one-way valve such as a BLOM-SINGER® voice prosthesis is inserted into the tracheoesophageal fistula. The one-way valve protects the airway during swallowing but opens under positive pressure from the trachea. The voice prosthesis, thus, permits a patient to divert air from the lungs into the esophagus and out through the mouth. Speech is created during passage of air through the upper part of the esophagus.

The prosthesis maintains the fistula open, transfers air from the trachea to the esophagus for voice production and prevents esophageal leakage into the trachea during swallowing. The oral cavity which extends into the throat has a high microbial population. However, the prosthesis being in contact with moisture in a warm, dark, nutrient rich environment is subject to growth of commonly found micro-organisms, typically Candida on the valve and the retaining flange. The microbial attack is currently being studied. The microbial attack organisms and sequence of events are quite complex and are still undetermined. The microbial growth on and into the soft silicone resin can interfere with function of the valve and can cause the flange to wrinkle and the valve to leak. The fouled device must be cleaned or discarded and replaced with a new device.

One type of current low pressure voice prosthesis can be removed by the patient every few days and can be replaced with a clean prosthesis. The removed prosthesis is soaked in hydrogen peroxide to sterilize and clean the valve and flange. Some patients however, have difficulty managing frequent removal and reinsertion of the prosthesis. Others, who are physically handicapped are not able to remove, sterilize, or reinsert the prosthesis.

A longer dwelling, low pressure voice prosthesis has been developed that can remain in place in the tracheoesophageal fistula for many weeks or months, depending on the patient and conditions of use. The patient can confidently use the prosthesis for longer periods. The longer dwelling voice prosthesis is not removable by the patient. Trips to a health care specialist to remove and replace the prosthesis are greatly extended providing increased comfort and lower cost to the patient.

Another type of soft voice prosthesis includes a rigid stiffening ring 14 inserted into a groove in the soft body of the prosthesis. Though the ring stiffens the body adjacent the valve it does not prevent distortion of the body by muscular movement or distortion of the valve by growth of yeast.

U.S. Pat. No. 5,578,083 issued Nov. 26, 1996, discloses the use of a stiff cartridge to support the soft silicone prosthesis and to provide a seat for the valve. However, microbial growth still proceeds to a point at which the valve can not be reliably sealed.

Microbial growth on the valve can also cause distortion of the shape of the valve or form wrinkles in the body of the valve which prevents the valve from closing. Leaking also appears to be due to distortion of the valve body adjacent to the seat of the valve and to microbial growth on the seat. Forming the valve with an arcuate dome shape increased resistance to folding or bending of the valve. However, some valves still leaked after extended placement in a fistula.

List of Prior Art

| U.S. Pat. No. | Patentee |
|---|---|
| 3,932,627 | Margraf |
| 4,054,139 | Crossley |
| 4,483,688 | Akijama |
| 4,563,485 | Fox, Jr. et al. |
| 4,581,028 | Fox, Jr. et al. |
| 4,603,152 | Laurin, et al. |
| 4,612,337 | Fox, Jr. et al. |
| 4,615,705 | Scales et al. |
| 5,019,096 | Fox, Jr. et al. |
| 5,567,495 | Modak et al. |
| 5,624,704 | Darouiche et al. |
| 5,772,640 | Modak et al. |
| 5,902,283 | Darouiche et al. |
| 6,083,208 | Modak et al. |
| 6,106,505 | Modak et al. |

### STATEMENT OF PRIOR ART

Margraf discloses the use of a silver-heparin-allantoin complex to a form non-thrombogenic, self sterilizing surface on prosthetic valves or arterial grafts. The complex can be coated or impregnated into the surface of the valve or graft.

Crossley coats the surface of an urinary tract catheter with Ag or Ag compounds by dispersing silver or its compound in resin. The surface is abraded to expose the silver material. The coating contains 10% by weight of silver (col 4, line 10). The coating can be extremely thin such as those deposited by electroless deposition (col 4, lines 16-18).

Fox, Jr. et al., 4,563,485 discloses use of silver norfloxacin or silver perfloxacin to render muscular graft prosthesis formed from resins such as silicone infection resistant.

Fox, Jr. et al., utilizes silver metal salts of sulfonamides or other antimicrobials for the same purpose.

Fox, Jr. et al., discloses and claims a method of preparing an infection resistant material by solvent impregnation of the material with a silver salt and another compound and reaction in situ to form a silver salt.

Scales et al., provides a bioerodible silver coating on the surface of endoprosthetic orthopaedic implants to render the surface antimicrobial.

Fox, Jr. et al., discloses the use of a complex of a silver salt and chlorhexidine to add antimicrobial properties to biomedical polymers such as silicones.

Laurin, et al. discloses mixing an oligodynamic material such as a salt of silver, gold, platinum, copper or zinc with a resin to form an antimicrobial coating for catheters.

Akijama coats an oligodynamically active silver, gold or copper salt on the periphery of a tubular catheter.

Modak et al. and Darouiche et al. discloses the use of triclosan to silicone medical devices such as catheters to inhibit microbial growth. By coating the agent onto the surface of the device or soaking the device in swelling agent and then in a solution containing triclosan to introduce triclosan into the device.

When soft prosthesis were compounded with antimicrobial agents such as silver compounds at a level which resists growth of microorganisms, it was discovered that the prosthesis was irritating to and/or toxic to tissue in contact with the prosthesis.

### STATEMENT OF THE INVENTION

It has been discovered in accordance with the present invention that antimicrobial agents can be compounded into parts of a prosthetic that are not in contact with tissue. The antimicrobial parts will be free of microbial growth for an extended period which contributes to longer use of the prosthesis in vivo. For example, the valve in most voice prostheses is not in contact with tissue. It is only in intermittent contact with body fluids. The same is true of the inside surface of the tubular prosthesis and/or the facial and inside surfaces of rings or cartridges present to reinforce the soft body of a prosthesis. By adding an amount of microbial agent effective to resist growth onto or into the valve, ring or cartridge it is found that microbial growth is delayed for a significant period without any evidence of irritation or toxicity to the tissue.

It also has been discovered that microbial growth on bio-compatible polymers such as silicone elastomers is complex. It is very difficult to duplicate the microbial growth in vivo. Furthermore, the microbes appear to be patient dependent. The growth proceeds through several stages as follows:

1. Deposition of a biofilm

2. Feeding and attack by microbes (bacteria origin)

3. Feeding and attack by yeast

The microbial growth on the surface of the polymer can be impeded by breaking the chain of any of the three events.

An antimicrobial surface on a prosthetic device can be provided by soaking the surface of the device in solution containing an agent that reacts with or interferes with the biofilm, microbes or yeast. Coating the surface of the device with a layer of the agent, grafting the agent onto the surface, treating the surface such as with high energy beams to render the surface antimicrobial and dispersing the agent within the wall of the device at a level at which the surface is antimicrobial are other methods of providing an antimicrobial surface.

The body of the prosthesis can be formed of a mixture of dissimilar materials such as elastomers or can be formed of layers of different elastomers. Reduced tendency of microbes to adhere to the surface of silicone elastomers can be provided by coating the surface of a silicone elastomer with a layer of polyurethane elastomer. The rate of leaching a dispersed antimicrobial material from an elastomer can be controlled by coating the surface of the elastomer with a layer impermeable to the antimicrobial material such as a phenyl silicone elastomer in the case of a silicone elastomer. The leach rate of the outer layer can be controlled by forming pores of controlled size in or apertures through the layer.

The isolation of the valve from tissue is enhanced by recessing the valve forward of the rearward edge of the prosthesis and/or forward of a flange which seats the prosthesis in a tracheoesophageal fistula.

The body of the prosthesis may have some antimicrobial properties as long as the surface of the body is not toxic to tissue. For example, the body can be formed of a polyurethane polymer which resists attachment of a biofilm or a microbial layer.

The antimicrobial agent can also be compounded by dispersion into the raw material. For example, silicone elastomer can contain up to 100 phr of an antimicrobial agent such as silver or silver compounds such as silver oxide suitably about 5 up to less than 50 phr of silver oxide. Other suitable antimicrobial compounds toxic to tissue can be used in the non-tissue contact regions such as gold, platinum, copper, zinc metal powder or oxides and salts thereof.

A metal coating that retards microbial growth can be deposited on a silicone surface by ion deposition in a vacuum chamber (SPIRE process) as disclosed in U.S. Pat. Nos. 5,474,797 and 5,520,664, the disclosures of which are incorporated herein by reference. Suitable metals are platinum, iridium, gold, silver, mercury, copper, iodine, and alloys, compounds and oxides thereof.

Preferred antimicrobial agents are organic antimicrobial agents that can be dispersed throughout the raw material preferably a food grade preservative such as an aromatic carboxylic acid or C₁ to Cq ester thereof such as butyl paraben butyl p-hydroxy benzoate or an alkene carboxylic acid salts such as alkali metal sorbate salt or a halahydroxy aromatic ether such as triclosan (2,4,4′-trichloro-7′hydroxydiphenyl ether.

The agents can be dispersed throughout the raw material by milling a dry powder into liquid resin before curing by predissolving in minimum amount of solvent and then mixing or milling the solution into the liquid resin or heating the agent above its melting temperature but below its decomposition temperature and mixing the molten material with the liquid resin before molding. The agents were chosen to be sufficiently robust to survive the molding process.

Valves and cartridges for voice prosthesis have been compounded with a dispersion of antimicrobial agents and were subjected to in vitro and in vivo testing. The valves and cartridges are found to exhibit significant inhibition of microbial growth.

These and many other features and attendant advantages of the invention will become apparent as the invention becomes better understood by reference to the following detailed description when considered in conjunction with the accompanying drawings.

### BRIEF DESCRIPTION OF THE DRAWINGS

FIG. 1 is a schematic view of a voice prosthesis installed in a tracheoesophageal fistula;

FIG. 2 is a view in section of an assembly of the valve, cartridge and body of an embodiment of the voice prosthesis of the invention;

FIG. 2a is an enlarged sectional view of the valve securing means shown in FIG. 2 for purposes of clarity;

FIG. 3 is a side view in elevation of the valve shown in FIG. 2;

FIG. 4 is a front view in elevation of the valve shown in FIG. 2;

FIG. 5 is a rear view in elevation of the cartridge shown in FIG. 2;

FIG. 6 is a view in section taken along line 6-6 of FIG. 5;

FIG. 7 is a view in section taken along line 7-7 of FIG. 6;

FIG. 8 is a bottom view in elevation of the cartridge illustrated in FIG. 2.

FIG. 9 is a top view in elevation of the prosthesis illustrated in FIG. 9; and

FIG. 10 is a view in section taken along line 10-10 of FIG. 9.

### DETAILED DESCRIPTION OF THE INVENTION

The invention will be illustrated by a long-dwelling voice prosthesis with hard cartridge and a soft body voice prosthesis, though it is applicable to any prosthetic or medical device disposed in a body cavity having an environment conducive to growth of micro-organisms such as Candida Albicans.

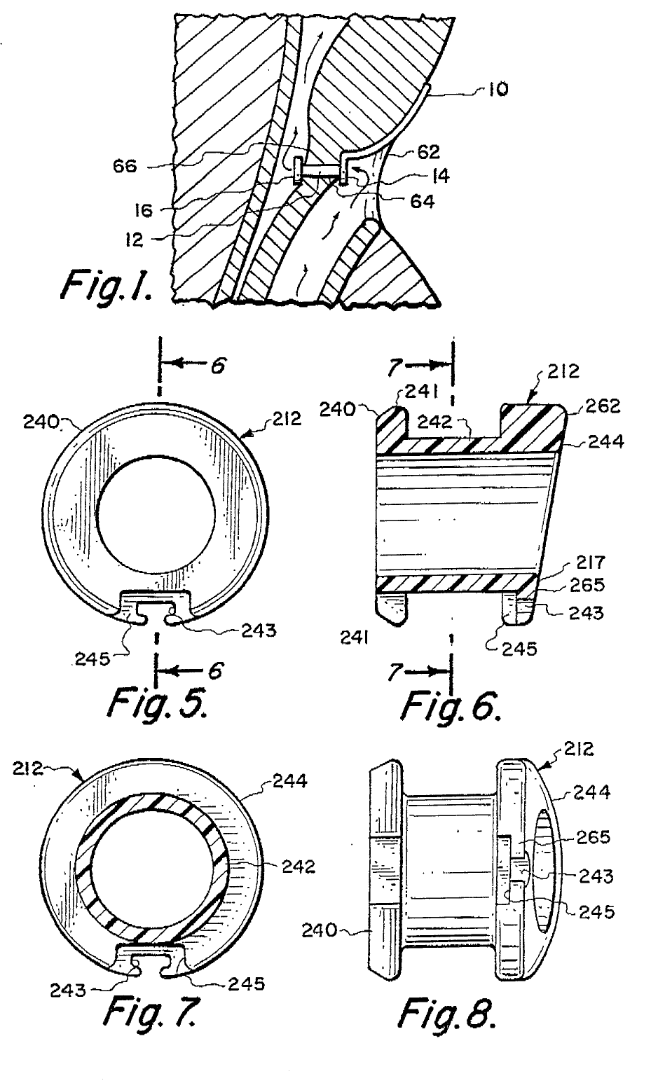

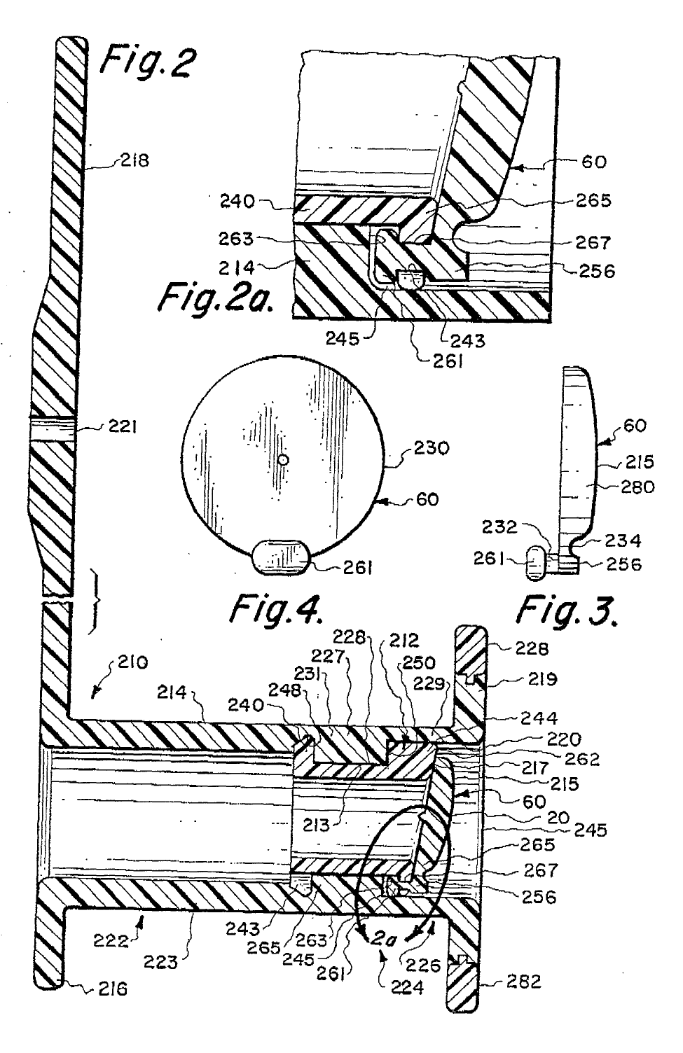

Referring now to FIGS. 1 and 8-9, a voice prosthesis 10 is shown inserted into a fistula 62 with the front flange 14 engaging the outer wall 64 of the trachea and the rear flange 16 engaging the wall 66 of the esophagus. The body 12 of the prosthesis 10 prevents the fistula 62 from closing. The body 12 and flanges 14, 16 are formed of an elastomer material which is non-toxic to tissue. The prosthesis 10, 310 also contains a valve 60, 360 as shown in FIGS. 2-4 and FIGS. 8, 9 which has an antifungal surface 215, 315 toxic to tissue. The valve 60, 360 is preferably separately molded and has a flap 20 or 2 posts 320 which are attached to the prosthesis 10, 310. In the soft prosthesis 310, the posts 320 are received in cavities 322 in the body and secured thereto by potting with a biocompatible adhesive such as RTV. The valve could also be mounted in a rigid sleeve attached to the distal end of the cartridge.

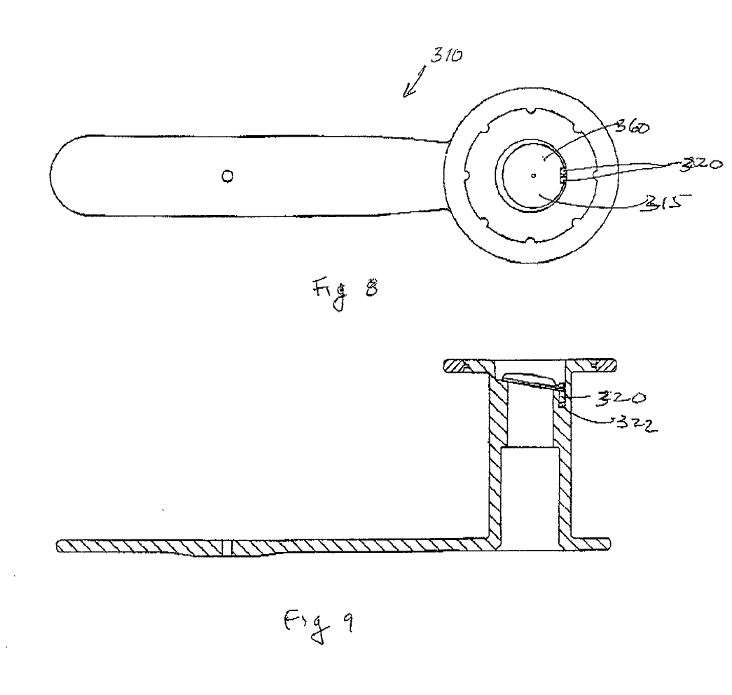

Referring again to FIGS. 2-4, a long dwelling prosthesis 210 can further contain an internal, rigid cartridge 212 which reinforces the body 214 of the soft prosthesis as shown in FIGS. 2-8 and as disclosed in U.S. Pat. No. 5,578,083 the disclosures of which are expressly incorporated herein by reference.

Referring particularly now to FIGS. 2 and 2a, a preferred voice prosthesis 210 is formed of a tubular body 214, a hollow, rigid cartridge 212 received in a channel 213 through the body 214 and a flapper valve 215 mounted on the rear face 217 of the cartridge 212.

A front tracheal flange 216 and a rear retention esophageal flange 219 are connected to the ends of the body 214. A flexible tab 218 can be attached to the front flange 216. The tab 218 can contain an aperture 221 which can be connected to an insertion tool, not shown. The body 214, front flange 216 and rear flange 219 are preferably a single molded, unitary structure formed from a biocompatible elastomer such as silicone resin, suitably a 50 durometer, medical grade, silicone elastomer. Since the resin is transparent and the prosthesis structure is small, the prosthesis is difficult to visualize and handle. Therefore, the molding resin generally, but not always, can contain a small amount, from 0.1 to 0.5% of a biocompatible pigment to aid in seeing the device. The pigment can be a heavy metal salt such as barium sulfate. The cartridge 212 can be formed of an inert, self-lubricating thermo plastic polymer, a fluorinated resin such as KYNAR, a semi-crystalline, low molecular weight polymer of vinylidine fluoride, such as TEFLON (polytetrafluoroethylene) or a polyalkylene resin such as polyethylene or polypropylene.

The tubular body 214 has a first section 222 having a wall 223 of a first thickness, a central section 224 having a wall 227 of a greater thickness and a third wall section 226 having a wall 229 of reduced thickness. The central wall section 224 forms a cylindrical boss 231 which is received in an annular channel 228 formed in the outer wall of the cartridge 212.

The hollow cartridge 212 has a front flange 240, a rear flange 244 forming a central channel 242 between the flanges 240, 244. The cartridge 212 is assembled with the body 214 by inserting the front flange 240 of the cartridge 212 into the rear opening 245 of the body 214 and forcing it through the central channel 213 of the body compressing the boss 231 until the front flange 240, seats against the end wall 248 of the boss 231 and the rear flange 244 seats against the rear wall 250 of the boss 231.

Referring now to FIGS. 3 and 4, the rear flange 244 has a horizontal slot 243 for receiving a tab 232 mounted on the front face of the valve 60 which communicates with an enlarged recess 245. The remaining volume in the recess 245 can be filled with biocompatible adhesive such as a silicone adhesive. Preferably, the tab 232 contains a bulbous end 261 which seats in the recess 245. The rear face 262 of the rear flange 244 can be angled to the vertical in order to preload the valve 60. Usually the angle is form 1 to 20 degrees, preferably 3 to 10 degrees.

Referring further to FIGS. 3 and 4, the flapper valve 60 has a round segment 230 connected to an attachment flap 256. A live hinge 234 in the form of a score line separates the segment 230 from the flap 256. A tab 232 is provided on the flap 256 for attaching the valve 60 to the body of the cartridge 212.

The hinge is located adjacent the lower, recessed portion of the rear face of the flange 244 which preloads the valve 60. The valve 60 is further strengthened by the increased thickness of the dome-shaped rear face 280 of the round segment 230. Leakage of the valve is further decreased due to the seating of the valve element 60 on the hard, smooth outer surface 217 of the rear flange 244 of the cartridge.

In order to assure that the rear flange 219 of the body 214 is fully seated on the esophageal wall surrounding a fistula, a narrow opaque ring 282 can be attached to or molded into the rear flange 219 as disclosed in U.S. Pat. No. 5,480,432 on Jan. 2, 1996, the disclosure of which is expressly incorporated herein by reference. An opaque pattern can also be provided by depositing opaque dots such as tantalum on the flange. The ring 282 has a width at least 10% the diameter of the rear flange usually from 10% to 50% the diameter of the annular rear flange. Usually the rear flange has a diameter of about 0.5 inch and the ring has a width of about 0.05 to 0.10 inch. The ring 282 preferably has an outer perimeter coincident with that of the rear flange 219 so that folds anywhere on the rear flange will be detected by the displayed image of the ring 282. The ring is preferably formed of the same flexible resin as the rear flange but contains an amount of radiopaque pigment such as barium sulfate sufficient to render the ring opaque to X-rays. Usually the pigment is present in an amount from at least 5% to 35%, generally around 20% by weight.

The front flange 240 of the cartridge 212 can have a bevel 241 so that it is easier to move the front flange 240 past the boss 231 on the body 214 of the device.

The body 214 can also contain a recess 220 placed forward of the rear flange 219 to further protect the valve from failing by further isolating the valve from contacting tissue. A hood may also be provided rearward of the flange 219.

Referring more particularly now to FIGS. 5-8 the front flange 240 and the rear flange 244 of the hard cartridge 212 may contain key shaped slots 243, 245 which cooperate with a key bar 265 on the bottom of the soft tubular body 214. The rear end 267 of the key bar 265 bears against the bulbous end 261 of the flexible tab 256.

Providing a microbial resistant valve according to the invention may eliminate or reduce the need to utilize a thick domed valve and a thicker, stiffer rear flange. Since the growth of a thick biofilm layer will be inhibited, warping of the valve is reduced or eliminated. The microbial resistant surface can be provided by coating or laminating a layer of microbial resistant polymer onto the exposed surfaces of the valve or by dispersing a microbial agent such as metal, metal oxide or salt or organic antimicrobial agent into the biocompatible resin.

The microbial resistant surface is formed of a polymer having a minimum thickness to inhibit microbial growth. The thickness of the coating can be as small as a monolayer and generally is at least 0.005 inches in thickness and usually from at least 0.01 to 0.3 inches in thickness. A layer of microbial resistant polymer can be coated onto the surface from solutions or dispersions of the polymer, can be formed on the surface by polymerization of monomers in situ, or by curing liquid prepolymers on the surface of the prosthesis. The layers can also be laminated to the surface by adhesive or by heat. In the case of certain elastomeric polymers that have the appropriate physical properties for use as a valve, the valve may be formed from the microbial resistant polymer.

The preferred manner of providing a surface resistant to microbial growth is to disperse the agent in the resin forming the portion of the device not in direct contact with body tissue. The agent can be inorganic such as a salt or oxide of silver, gold, platinum, zinc or copper, preferably silver oxide or organic materials soluble or dispersible in the resin forming the valve or the cartridge such as hydroxy aromatic carboxylic acids, esters thereof or halogenated phenols. The agent is present in the resin or at least in a surface layer in an amount effective to deter microbial growth and at a concentration that can be toxic to tissue. The portions of the device in contact with tissue can contain a much lower concentration of the microbial agent at a level non-toxic and non-irritating to tissue.

For example, in the case of silver oxide, the concentration of silver oxide effective to deter growth of microbial biofilm is from 1 to 50 phr, preferably 8 to 25 phr. The body of the device which is in direct contact with tissue can be compounded to include from 0.1 to 2 phr, preferably 0.5 to 1.0 phr of silver oxide.

The following experiments were conducted to determine the biocompatibility requirements of compounding silver oxide into bodies and valves of voice prosthesis at different concentrations and of coating the outside surfaces of a voice silicone elastomer prosthesis and valve with vaporized metal coatings by the SPIRE® process. The silver oxide was dispersed in the resin, molded to form a soft voice prosthesis body valve or disc and then cured. The silicone parts were tested in solution proportional to their size.

Cytotoxicity testing was performed on various concentrations of silver oxide and silicone elastomer, and on various combinations of bodies and valves. MEM (Minimum Essential Medium) Elution and Agarose Overlay tests were done. It was decided that the most applicable test, given the use of the voice prosthesis, is the MEM test, as it tends to be more sensitive. The Agarose overlay test is useful to help determine comparative degrees of toxicity for the different percentages of silver oxide.

TESTS PERFORMED

| | AGAROSE | |
|---|---|---|
| MATERIAL | OVERLAY | MEM |
| 14% Ag₂O | | |
| Q7-4750 w/14% Ag₂O (valves) | | Nontoxic |
| 10% Ag₂O | | |
| 10% Ag₂O sample discs | Toxic | |
| 10% Silver sample discs | Nontoxic | |
| Q7-4750 w/10% Ag₂O (10 units) | | Nontoxic |
| Q7-4750 w/10% silver (10 units) | | Nontoxic |
| Q7-4750 w/10% Ag₂O (valves) | | Nontoxic |
| Q7-4750 w/10% Ag₂O (bodies) | Toxic | Toxic |
| Q7-4750 body w/10% Ag₂O valve | | Nontoxic |
| 1.0% Ag₂O bodies/10.0% valves | | Intermediate |
| 0.5% Ag₂O bodies/10.0% valves | | Nontoxic |
| 8% Ag₂O | | |
| 0.5% Ag₂O bodies/8.0% valves | | Nontoxic |
| 5% Ag₂O | | |
| Q7-4750 body w/5% Ag₂O valve | | Nontoxic |
| Q7-4750 w/5% Ag₂O (bodies) | Toxic | Toxic |
| 2% Ag₂O | | |
| Q7-4750 w/2% Ag₂O (bodies) | Toxic | Intermediate |
| 0.5% Ag₂O | | |
| Q7-4750 w/0.5% Ag₂O (bodies) | Nontoxic | Nontoxic |
| 2% Gentian Violet | | |
| Q7-4750 w/2% Gentian | | |
| violet (discs) | Toxic | |
| 2% Copper Oxide | | |
| Q7-4750 bodies w/2% | | |
| Copper Oxide valves | Nontoxic | Nontoxic |
| Q7-4750 control | | |
| Q7-4750 bodies w/2% Copper | | |
| Oxide valves | | Nontoxic |

The tests showed that 10% silver oxide could be used in the valves if the bodies were straight silicone elastomer, or contain a very low percentage of silver oxide. However, the 10% silver oxide valves seems to be the upper end of toxicity.

The bodies and valves at 10% showed different results. They were tested in solution proportional to their size (theoretically), yet the bodies consistently showed a more toxic response than the valves. A theory is that the bodies simply had a greater mass even when this was compensated for in choosing the solution size, so more silver oxide was able to leach out into the test medium.

Based on these tests, in-vitro test discs were prepared using 5% and 10% silver oxide concentrations, and the clinical voice prosthesis units were prepared using 10% silver oxide valves.

Eight tests were performed using valves of different materials to test for measurable zones of inhibition. Sample discs were prepared of the various materials in the concentrations to be tested. The silver oxide, silver, copper, copper oxide, metallic copper, and gentian violet materials were mixed with silicone elastomer, in the concentrations listed. The SPIRE silver (SPIRE A and B), SPIRE Titanium, SPIRE copper were coatings on the valve using SPIRE's coating method. The novatran is a parylene coating and the BSI is a polyacrylamide coating, both done on the valves.

Cultures of Candida albicans were grown up for each test date. The Candida cultures were swabbed onto media plates and the sample discs were placed on the plates. The plates were incubated at the specified controlled temperature for 15-24 hours and the plates read for inhibition zones. The plates were then returned to the incubator until overgrown.

All tests were performed under the Class 100 laminar flow bench. Particle counting was performed on the clean bench prior to initiation of the testing.

Summary: Measurable zones of inhibition were demonstrated only on silver oxide, in both the 5% and the 10% concentrations, and on the 2% gentian violet. The zones of inhibition were consistently in the range of 5-7 mm around the test disc.

TESTING PERFORMED

| 1. | |
|---|---|
| Test Samples | Inhibition |
| 10% Ag₂O | Yes |
| 5% Ag₂O | Yes |
| 10% silver | |
| 5% silver | |
| Novatran | |
| BSI | |
| Q7-4750 (control) | |
| 2. | |
| Test Samples | Inhibition |
| New 10% Ag₂O | Yes |
| Old 10% Ag₂O | Yes |
| New 6% silver | |
| Q7-4750 (control) | |
| Old 10% silver xx | |
| xx Old Ag₂O was taken from a bottle past the expiration date | |
| 3. | |
| Test Samples* | Inhibition |
| Ag₂O soaked in | Yes |
| saline 1 week | |
| SPIRE A | |
| SPIRE B | |
| *Candida successfully rinsed off Ag₂O sample, but not off SPIRE samples | |
| 4. | |
| Test Samples* | Inhibition |
| 0.5% Ag₂O | |
| 1.0% Ag₂O | |
| 5% Ag₂O | |
| Q7-4750 (control) | |
| 10% Ag₂O | |

*No inhibition; dilutions done incorrectly.
(SPIRE A is a very hydrophilic surface with moderately smooth surface; SPIRE B is a moderate improvement in surface energy with a very smooth surface)

| 5. | |
|---|---|
| Test Samples | Inhibition |
| 5% Cu | |
| 2% Cu | |
| 1% Cu | |
| 10% Ag₂O | Yes |
| Q7-4750 (control) | |
| 5% Ag₂O, soaked in | |
| saline for 18 weeks | |
| 0.5% Ag₂O | |

| | |
|---|---|
| 6. | |
| Test Samples | Inhibition |

No data recorded (when lab accident occurred)

| 7. | |
|---|---|
| Test Samples | Inhibition |
| 10% Ag₂O | Yes |
| 2% copper oxide | |
| SPIRE Ti | |
| SPIRE Cu | |
| 5% Metallic Cu | |
| Domed valve, Q7-4750 | |

| 8. | |
|---|---|
| Test Samples | Inhibition |
| 2% Gentian violet | Yes |
| SPIRE gold | |
| SPIRE Titanium | |
| Q7-4750 (control) | |

Based on the cytoxicity information and the results of the in-vitro tests, it was decided that the clinical units of the silicone elastomer bodies and 10% silver oxide valves, and SPIRE-coated bodies with 10% silver oxide valves would be clinically tested.

Ten patients were given the clinical units under supervision.

STUDY RESULTS

| Patient | Control | | Clinical Unit | | | | |
|---|---|---|---|---|---|---|---|
| Number | Time | | Time | | Increase | | Device |
| 1 | 8 | days | 49 | days | 41 | days | 10% Ag₂O |
| | 27 | days | 36 | days | 9 | days | SPIRE-coated |
| | | | | | | | Ag₂O |
| 2 | 59 | days | 127 | days | 68 | days | 10% Ag₂O |
| | 36 | days | | | | | |
| 3 | 28 | days | 69 | days | 41 | days | 10% Ag₂O |
| | | | 215+ | days | 187 | days | 10% Ag₂O |
| 4 | 22 | days | 258+ | days | 236 | days | SPIRE-coated |
| 5 | 35 | days | 42 | days | 7 | days | 10% Ag₂O |
| | 61 | days | 26 | days | | | |
| 6 | 42 | days | 13 | days | −29 | days | 10% Ag₂O |
| | 42 | days | 33 | days | −9 | days | 10% Ag₂O |
| | | | 38 | days | −4 | days | 10% Ag₂O |
| 7 | | | 26 | days | | | 10% Ag₂O |
| | | | 4 | days | | | Ag₂O valve/ |
| | | | | | | | SPIRE |
| | | | 10 | days | | | SPIRE-coated |
| 8 | | | 222 | days | | | 10% Ag₂O |
| 9 | | | 296 | days | | | 10% Ag₂O |
| 10 | | | 98 | days | | | 10% Ag₂O |
| | | | 287+ | days | | | 10% Ag₂O |

The 27 day sample used by patient #1 had the body SPI-Silicone coated to change its surface characteristics and the valve was silver oxide. Patient #4 used a voice prosthesis which had the body SPI-Silicone coated. Patients #8, #9 and #10 used voice prosthesis with standard silicone bodies and 10% concentration silver oxide valves.

Organic antimircobial are preferred since they are more readily and evenly dispersed in resin in amounts usually from 0.2 to 5 percent by weight.

Preferred Percentages of Additives:

| | |
|---|---|
| Triclosan | 0.25 to 2.0% |
| Butyl paraben | 0.25 to 1.0% |

Methods of Introduction:

There are three main methods of introduction of organic additives into the silicone elastomer material. The first method is simply mixing or milling the additives as a powder into silicone elastomer. This is done to either part of a two-part silicone elastomer system or to both parts together prior to molding. The problem with this method is the complete dispersion of the additive in the silicone.

The second method of introduction of the additive into silicone is to pre-dissolve the additive in a minimal amount of isopropanol or other appropriate solvent. This liquid mixture is then mixed or milled into the silicone as described above. The advantage to this method is that there is better dispersion of the additive within the silicone. One disadvantage is that it has been found that the addition of isopropanol negatively affects the physical properties of the finished, cured silicone. These effects are proportional to the amount of isopropanol added and can be minimized to negligible by the addition of only the minimal required isopropanol.

The third method is preferred as it allows dispersion of the additive throughout the silicone without the use of a solvent. This method is simply heating the additive above its melting temperature, but not past the decomposition temperature. It is then mixed into half of the two part silicone at this temperature. The silicone is allowed to cool prior to mixing both parts together and molding. This method provides a uniform distribution of additive throughout the silicone matrix.

Toxicity testing:

| Test | Results |
|---|---|
| Triclosan: | |
| Cytotoxicity (MEM Elution) on the valve material alone | Toxic |
| Cytotoxicity (MEM Elution) on the device with | Nontoxic |
| valve material | |
| Acute Oral Toxicity (7 day observation in the mouse) | Nontoxic |
| Butyl Paraben | |
| Cytotoxicity (MEM Elution) on the device with | Nontoxic |
| valve material | |
| Acute Oral Toxicity (7 day observation in the mouse) | Nontoxic |

Zone of Inhibition Data

Preface:

In each of these cases, unless noted, a sample of silicone with respective additive was punched from a slab. The final dimensions of the sample pieces were roughly 5 mm in diameter by 2 mm thick. Each variation of these samples were placed separately in 0.45 saline solution and incubated at 37° C. At the specified time, a sample was taken out of the solution and evaluated for zone of inhibition.

In each of the cases below, the testing organisms was Candida albicans, ATCC 10231. The medium used was Sabouraud Dextrose agar. Incubation time was 18 to 24 hours at 37° C. The zone of inhibition test was performed per internal testing standards of Helix Medical, Inc. The base material was Nusil MED 4940 silicone.

The zone of inhibition test technically measures leachability of an antimicrobial from a test article. The samples are placed on a lawn of microbial organisms of choice. As the substance leaches from the test sample, there is a concentration gradient set up as a function diffusion through the sample and diffusion away from the sample. At a certain concentration, a critical concentration, the growth of microorganisms will be greatly reduced. This is manifest as no growth or greatly reduced growth in a radius around the sample. With the purpose of the invention in mind, the size of the zone of inhibition is relatively unimportant, as long as the longevity of the substance with some microbial activity is maintained over time in a soaking condition. The result chosen to signify acceptable antimicrobial activity is inhibition of microbial growth underneath the sample. This signifies that the concentration at the surface of the sample has retained at least the critical concentration of antimicrobial substance. If the surface concentration can be maintained at or above the critical concentration, then little to no growth will colonize on the surface this material.

Note: For inhibition underneath sample, it is measured either as Inhibition (I), Partial Inhibition (PI), or No Inhibition (NI).

| | | |
|---|---|---|
| | | Inhibition |
| Time Soaked | Zone of | Underneath Sample |
| (weeks) | Inhibition (mm) | (I, PI, NI) |

Triclosan 0.5%:

| | | | |
|---|---|---|---|
| 1 | day | 0 | I |
| 1 | | 0 | I |
| 2 | | 0 | I |
| 3 | | 0 | I |
| 4 | | 0 | I |
| 12 | | 0 | I |
| 16 | | 0 | I |

Triclosan 1.0%:

| | | | |
|---|---|---|---|
| 1 | day | 1 | I |
| 1 | | 1 | I |
| 2 | | 1 | I |
| 3 | | 1 | I |
| 4 | | 1 | I |
| 8 | | 1 | I |
| 12 | | 1 | I |
| 16 | | 0.5 | I |

Triclosan 2.0%:

| | | | |
|---|---|---|---|
| 1 | Day | 1 | I |
| 1 | | 1 | I |
| 2 | | 1 | I |
| 3 | | 1 | I |
| 11 | | 1 | I |
| 12 | | 1 | I |
| 16 | | 1 | I |
| 20 | | 1 | I |
| 24 | | 1 | I |

Triclosan 2.0% molded valves:

| | | | |
|---|---|---|---|
| 1 | day | 1 | I |
| 1 | | 1 | I |
| 2 | | 1 | I |
| 3 | | 1 | I |
| 4 | | 1 | I |
| 8 | | 1 | I |

Butyl paraben 1%:

| | | | |
|---|---|---|---|
| 1 | Day | 1.5 | I |
| 1 | | 1.5 | I |
| 2 | | 1.5 | I |
| 3 | | 2 | I |
| 4 | | 2 | I |
| 8 | | 2 | I |
| 12 | | 1 | I |
| 16 | | 1 | I |

Butyl Paraben 1% molded valves:

| | | | |
|---|---|---|---|
| 1 | Day | 0 | I |
| 1 | | 0 | I |
| 2 | | 0 | I |
| 3 | | 0 | I |
| 4 | | 0 | I |
| 8 | | 0 | PI |

Voice prosthesis formed with microbial resistant valves will be able to be used for much longer periods without the need to remove able prosthesis for cleaning. The prosthesis can be made with thinner valves, body and flanges since there is no need to be as stiff and rigid to avoid bending and wrinkling due to growth of Candida Albicans. The body of the voice prosthesis can also be compounded with antimicrobial agents at a level acceptable to the FDA.

The Indwelling Low Pressure Voice Prosthesis of the invention is designed for those persons who are unable or resistant to changing the voice prosthesis every two or three days as was recommended for the non-indwelling, patient-removable Low Pressure Voice Prosthesis. The Indwelling Low Pressure Voice Prosthesis has been specifically designed to maintain the placement of the prosthesis in the tracheoesophageal puncture so that routine changing of the device is not necessary.

It is to be realized that only preferred embodiments of the invention have been described and that numerous substitutions, modifications and alterations are permissible without departing from the spirit and scope of the invention as defined in the following claims.

1. A prosthetic device for use in contact with animal tissue comprising in combination:
a first body portion having a first surface portion in direct contact with said tissue; and
a second body portion having a second surface not in direct contact with said tissue;
said second surface having anti-microbial activity at a level greater than said first surface.

2. A prosthetic device according to claim 1 in which the second surface is in fluid contact with said tissue.

3. A prosthetic device according to claim 2 in which the second surface is in intermittent fluid contact with said tissue.

4. A prosthetic device according to claim 1 in which the first surface exhibits antimicrobial properties at a level non-toxic and non-irritating to tissue.

5. A prosthetic device according to claim 1 formed of a biocompatible material.

6. A prosthetic device according to claim 5 in which the material comprises a medical grade silicone elastomer.

7. A prosthetic device according to claim 6 in which the first body portion comprises a tubular voice prosthesis having a central channel and the second body comprises a valve, ring or cartridge mounted within said channel and in which the outer surface of the tubular voice prosthesis is the first surface and the outer surface of the valve or the inner surface of the cartridge or ring is the second surface.

8. A prosthetic device according to claim 1 in which the second surface has antimicrobial properties provided by a coating applied to said second surface or by dispersing or absorbing antimicrobial agents in the material forming said second body portion.

9. A prosthetic device according to claim 5 in which the antimicrobial agent is selected from the group consisting of inorganic particles of metals having antimicrobial properties and oxides, salts and halides thereof and an organic antimicrobial material.

10. A prosthetic device according to claim 5 in which the organic antimicrobial material is selected from unsaturated alihatic acid salts thereof, halogenated hydroxy aromatic acids, esters thereof and aromatic ethers.

11. A prosthetic device according to claim 9 or 10 in which the metal, salt or oxide thereof is present in an amount from 5 to 50 phr and the organic antimicrobial material is present in an amount from 0.2 to 5% by weight.

12. A prosthetic device according to claim 10 in which the organic antimicrobial material selected from the group consisting of butyl paraben and triclosan.

13. A prosthetic device according to claim 9 in which the inorganic particles comprise silver oxide.

14. A method of increasing the life of a valve having an outside surface not in contact with tissue or a cartridge or a ring having an inside surface not in contact with tissue disposed in a channel through the body of a voice prosthesis, the outside surface of the body being in direct contact with tissue comprising the steps of:
providing antimicrobial activity on the outside surface of the valve or cartridge at a level that is effective to retard detrimental microbial growth; and
disposing the valve or cartridge within said channel, said body having an outside surface that is non-irritating and/or non-toxic to said tissue.

15. A method according to claim 14 in which the microbial surface is provided by dispersing the antimicrobial agents in at least one of the valve, cartridge or ring.

16. A method according to claim 14 in which microbial growth on said surface proceeds through the steps of:
1. Depositing a biofilm;
2. Feeding and attack by microbes of bacteria origin;
3. Feeding and attack by yeast;
and said antimicrobial activity is provided by inhibiting or interfering with at least one of said steps.

17. A voice prosthesis for use in contact with tissue comprising in combination:
a body having a central channel and an annular wall having an inside surface and an outside surface;
a valve having an inside surface and an outside surface mounted to seal and intermittently open said channel; and
the outside surface of the valve not being in contact with tissue and having antimicrobial properties such that tissue in contact with said outside surface is irritated by and/or toxic to said surface.

18. A voice prosthesis according to claim 17 in which the outside surface of the body is in contact with tissue and the outside surface of the body is non-irritating to and non-toxic to said tissue.

19. A voice prosthesis according to claim 18 in which said body and said valve are formed of an elastomer.

20. A voice prosthesis according to claim 19 in which the valve is formed of a silicone elastomer containing a dispersion of or outside coating of an antimicrobial material.

21. A voice prosthesis according to claim 20 in which the antimicrobial material is selected from metal and salts and oxides thereof and organic antimicrobial materials.

22. A voice prosthesis according to claim 21 in which the material is selected from silver oxide in an amount from 6 to at least 50 phr and butyl paraben and triclosan in an amount from 0.2 to 5% by weight dispersed in the resin forming the valve.
